(19) United States
(12) Patent Application Publication
(10) Pub. No.: US 2015/0327969 A1
(43) Pub. Date: Nov. 19, 2015

(54) VETERINARY MODULAR BODY WRAP

(71) Applicant: Therese Leigh Entler, Vancouver, WA (US)

(72) Inventor: Therese Leigh Entler, Vancouver, WA (US)

(21) Appl. No.: 14/277,193

(22) Filed: May 14, 2014

(51) Int. Cl. A61D 9/00; A61F 7/08; A61D 7/00

(52) U.S. Cl. CPC A61D 9/00; A61D 7/00; A61F 7/08

(57) ABSTRACT

A veterinary modular body wrap for positioning and holding a medical dressing, hot pack, cold pack, or other therapeutic article on a desired wound site or other location on the body of an animal providing protection and/or therapy as well as breathability and moisture control at the wound or therapy site.

### BACKGROUND OF THE INVENTION

1. Field of the Invention

The present invention generally relates to a veterinary modular body wrap comprising two or three modular units which may be used to cover one or more of a feline or canine chest area, midriff area, and hindquarters area. More particularly the present invention relates to a veterinary modular body wrap providing a platform for a medical dressing useful in treatment and/or healing of post—surgical and post—nonsurgical injury therapeutic use. Still more particularly the present invention relates to a veterinary modular body wrap that is breathable to allow air in and moisture out thereby helping in the healing or treatment of an injury further having a reusable fixing means for securely positioning said dressing wrap substantially preventing displacement or removal of said dressing wrap by the animal or its movements. Furthermore the present invention relates to a veterinary modular body wrap into which one or more dressings or medical sponges may be removably attached to the interior surface of said dressing wrap and/or heating/cooling packets may be removably attached to either interior or the exterior surface of said veterinary dressing wrap to help promote the treatment and/or healing of post—surgical and post—nonsurgical conditions.

2. Description of the Related Art

Traditionally post—surgical and nonsurgical post—injury bandaging utilizes a gauze tape wrapped around a portion of the pet's anatomy, said gauze tape, and then secured in place by adhesive tape. This regimen requires that said gauze tape and adhesive tape be replaced every time the dressing needs changing, requiring large amounts of bandaging materials and recreating large amounts of medical waste to be disposed of. Additionally, applying this system of bandage can easily reduce blood circulation to the wound or injury site if not properly applied. This makes changing dressings by someone unfamiliar with first aid difficult. Further, because most adhesive tapes used for wound dressing are made of non-stretchable materials they can be uncomfortable for the feline or canine which can encourage them to try to chew or scratch the dressing off. This discomfort and the resulting attempts to remove the bandaging required the use of an E-collar or cone.

One solution to the limitations of the traditional bandaging regimen has been the use of a one piece stretchable fabric body wrap. This type of reusable, washable, stretchable full body wrap is exemplified, for example, by the Surgi Snuggy brand body wrap sold by Hamilton Biovet.

Another such whole body wrap which is reusable and used to calm a pet by means of applying pressure at various acupressure points as disclosed for example, in U.S. Pat. No. 6,820,574 issued Nov. 23, 2004 to Sharpe.

While such full body wraps are an improvement over traditional gauze and surgical tape bandaging they require that the whole feline or canine body including chest, midriff, and hindquarters be covered. When only one portion of the pet's anatomy has been affected by surgery or injury there is no need for such complete body coverage. Additionally, this full body type wrap can be more uncomfortable than is necessary for the recovery of the pet.

The use of wound covering veterinary wraps for animals, particularly pets, most particularly dogs and cats, for protection from weather and/or contamination is known. Some are waterproof or water resistant, and some are neither. However, none of them address the ability to removably mount a medical dressing as well as provide moisture removal from the wrapped site to promote healing and comfort. Thus, there remains an unfilled need to provide a post-surgical and post injury therapeutic body wrap that can accommodate covering only that portion of the feline or canine body requiring a post-surgical or post injury dressing that can provide the ability to removably secure medical dressings and the like over a wound or injury site as well as removably secure hot and cold packs where needed.

### DISCLOSURE OF THE INVENTION

The present invention provides advantages and alternatives over the prior art by providing a veterinary modular body wrap comprising three modular units for positioning and holding a medical dressing or therapeutic article in at least one desired location on an animal body comprising in cooperative combination: a modular center unit dimensioned to lay across the back of an animal body comprising four-way stretch fabric materials having an unbroken loop (UBL) fabric layer on both outer surfaces with an open cell polyurethane center layer; a modular chest unit dimensioned to provide a chest covering portion comprising four-way stretch fabric materials having an unbroken loop (UBL) fabric layer on both outer surfaces with an open cell polyurethane center layer, removably attachable to said center unit by modular chest unit attachment portions having removably securing portions comprising the hook part of a hook-and-loop connection; and a modular hindquarters unit dimensioned for covering the canine or feline hindquarters comprising four-way stretch fabric materials having an unbroken loop (UBL) fabric layer on both outer surfaces with an open cell polyurethane center layer, removably attachable to said center unit by securing portion positioned over the hindquarters comprising the hook part of a hook-and-loop connection, and a tail bore allowing the animal's tail to pass through said modular hindquarters unit; thereby creating a desired modular body wrap consisting comprising three modular units for positioning and holding a medical dressing or therapeutic article in desired positions on an animal's body while eliminating the need of a “cone” surrounding the animal's head.

According to a further aspect of the present invention there is provided a veterinary modular body wrap comprising two modular units for positioning and holding a medical dressing or therapeutic article in at least one desired location on an animal body comprising in cooperative combination: a modular chest unit dimensioned to provide a chest covering portion comprising four-way stretch fabric materials having an unbroken loop (UBL) fabric layer on both outer surfaces with an open cell polyurethane center layer, removably attachable to said animal body by modular chest unit attachment portions having removably securing portions comprising the hook part of a hook-and-loop connection; and a modular hindquarters unit dimensioned for covering the canine or feline hindquarters comprising four-way stretch fabric materials having an unbroken loop (UBL) fabric layer on both outer surfaces with an open cell polyurethane center layer, removably attachable said animal body by securing portion positioned over the hindquarters comprising the hook part of a hook-and-loop connection, and a tail bore allowing the animal's tail to pass through said modular hindquarters unit; thereby creating a desired modular body wrap consisting comprising two modular units for positioning and holding a medical dressing or therapeutic article in desired positions on an animal's body while eliminating the need of a “cone” surrounding the animal's head.

Yet another aspect of the present invention is the elimination of the need to use a “cone” surrounding the animal's head to prevent the animal from dislodging any applied medical dressing or therapeutic article.

According to yet another aspect of the present invention there is presented a pocketed securing tab portion providing for easier un-securing without the need of tabs.

According to a yet further aspect of the present invention there is provided a two or three unit modular veterinary body wrap which allows for the use of individual modular units separately to provide the body wrap necessary for the welfare of the animal.

According to still another aspect of the present invention there is the ability to provide comfortable post-injury, post-surgical, and therapeutic care to an animal while also providing protection and moisture control to the wound site.

According to still yet another aspect of the present invention the present invention provides for a reusable modular veterinary body wrap that does away with the need for replacing gauze and surgical tape every time the dressing and/or therapeutic article such as, for example, a hot or cold pack requires replacement.

The present invention thus advantageously provides an easy to use, animal comfortable, economical, reusable veterinary body wrap.

### BRIEF DESCRIPTION OF THE DRAWINGS

FIG. 1 shows a right side view of one preferred embodiment of veterinary modular body wrap of the present invention.

FIG. 2A shows a right side view of a non-self-securing embodiment of one preferred center unit of the veterinary modular body wrap of the present invention.

FIG. 2B shows a right side view of a self-securing embodiment of one preferred center unit of the veterinary modular body wrap of the present invention.

FIG. 3 shows a right side view of a self-securing chest unit of the veterinary modular body wrap of the present invention.

FIG. 4 show a right side view of a self-securing hindquarters unit of the veterinary modular body wrap of the present invention.

FIG. 5 shows a plan top view of a non-self-securing embodiment of one preferred chest unit of the veterinary modular body wrap of the present invention.

FIG. 6 shows a plan top view of a non-self-securing hindquarters unit of the veterinary modular body wrap of the present invention.

FIG. 7 shows a plan top view of a non-self-securing embodiment of one preferred center unit of the veterinary modular body wrap of the present invention.

FIG. 8 shows a perspective end view of lift pocket hook portion end tab suitable for use with all units of the veterinary modular body wrap of the present invention.

### DESCRIPTION OF THE PREFERRED EMBODIMENTS OF THE INVENTION

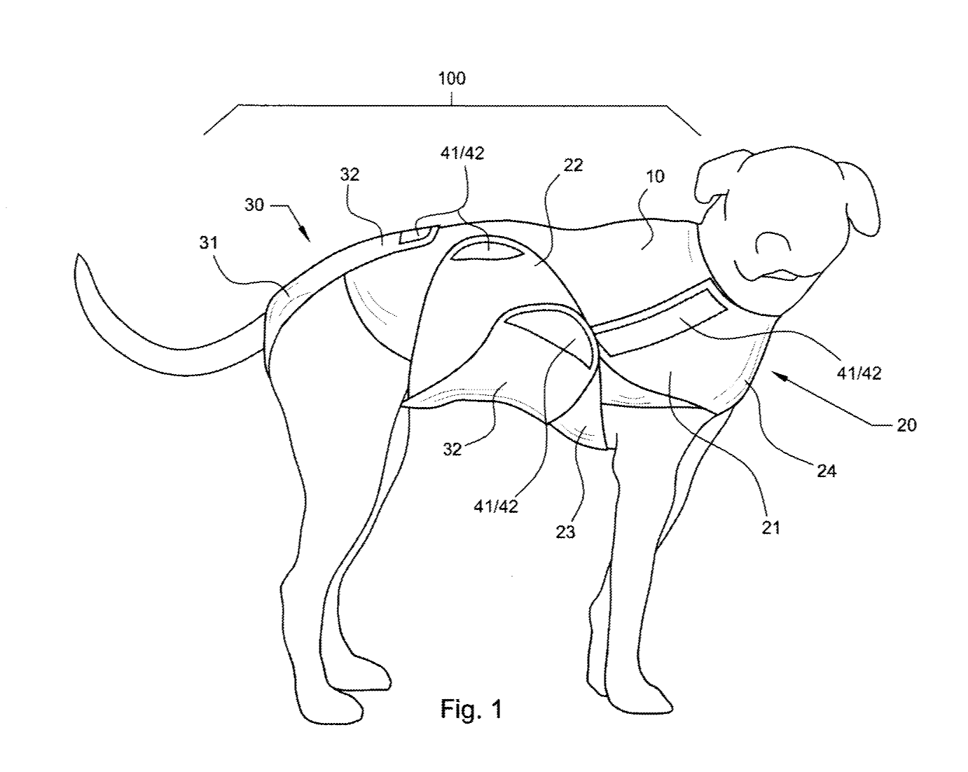

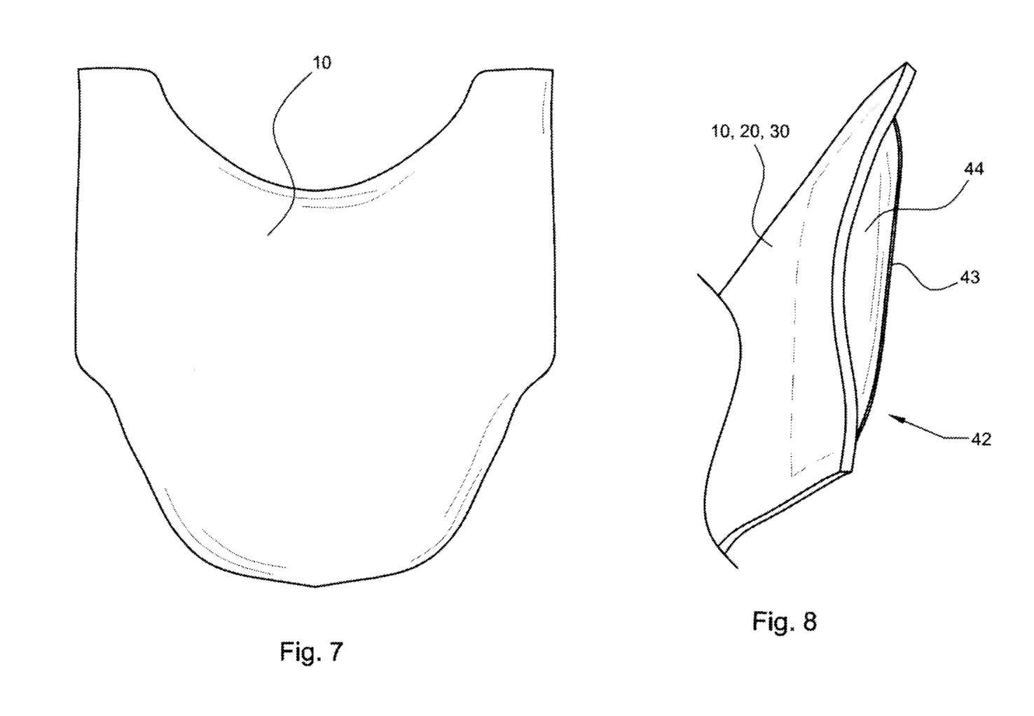

Reference will now be made to the drawings, wherein to the extent possible like reference numerals are utilized to designate like components throughout the various views. Referring to FIG. 1, which presents a right side view of one preferred embodiment of the claimed veterinary modular body wrap 100 comprising a center unit 10 dimensioned to drape over a canine or feline back in a manner similar to a saddle blanket on an equine, and not being self-secured around said canine or feline (also see FIG. 7).

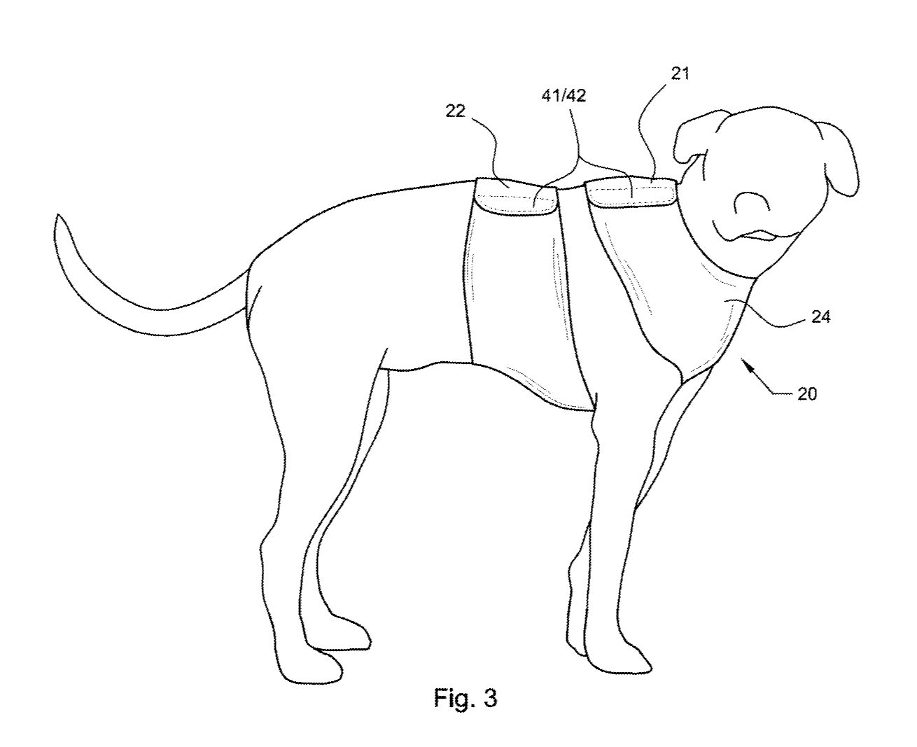

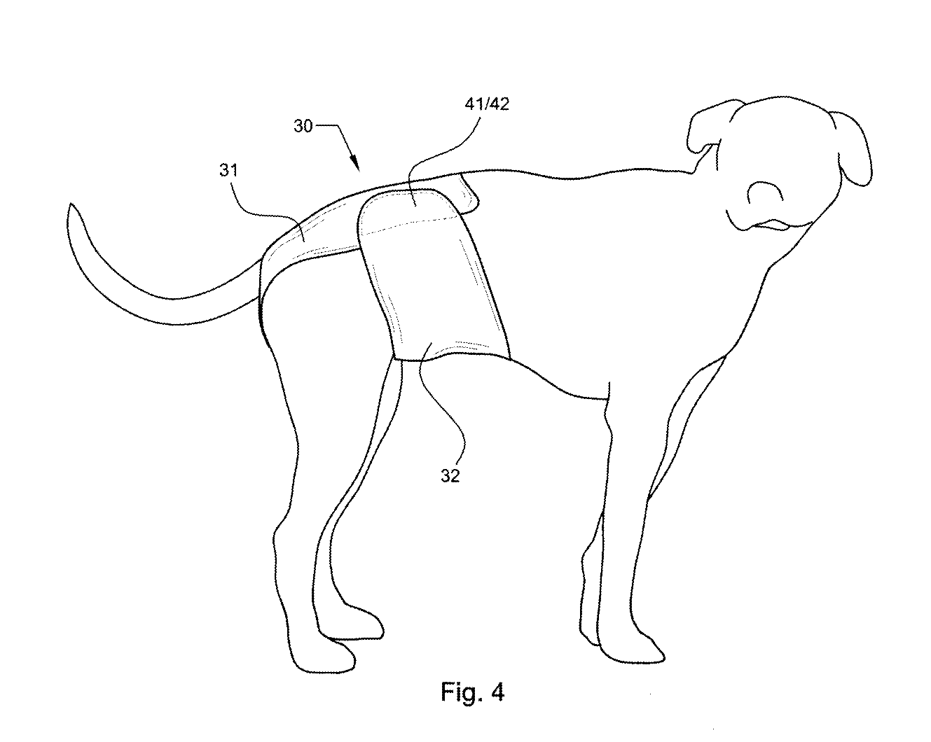

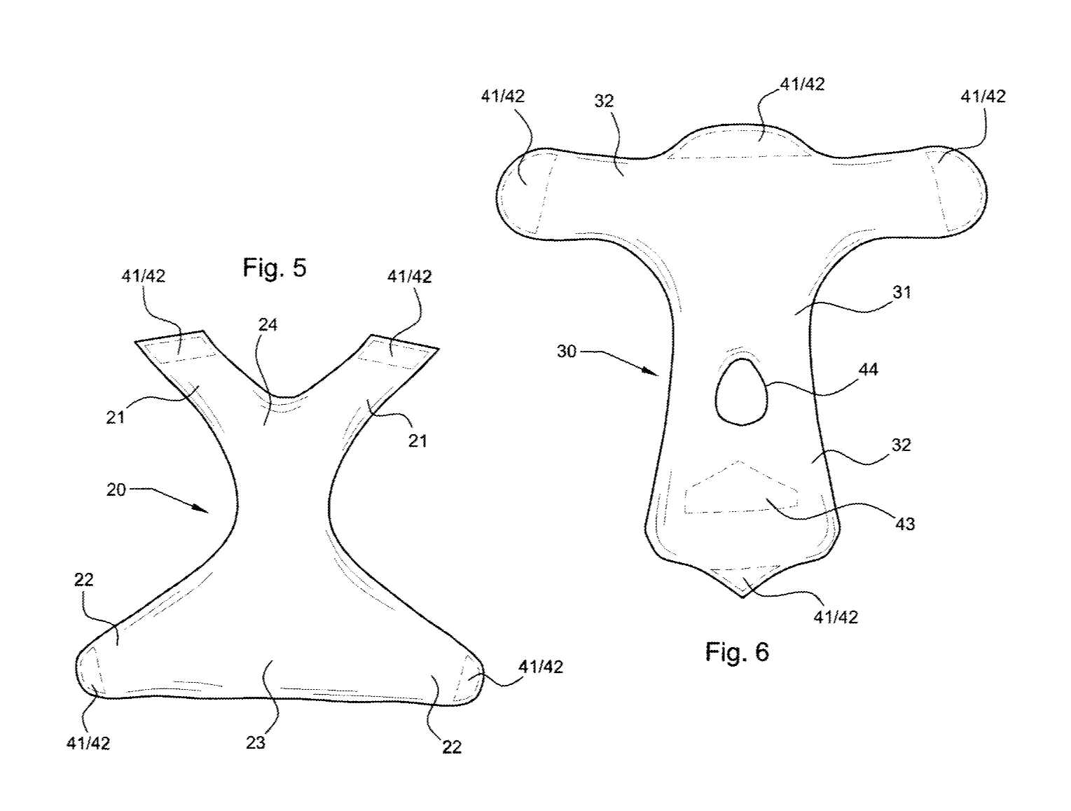

As further shown in FIG. 1 there is presented one preferred embodiment of a chest unit 20 having a chest covering portion 23, a chest covering mounting portion 24 removably attachable to said center unit 10 by means of chest covering attachment portions 21 and 22 (see also FIG. 5). Also shown are removably securing portions 41 or 42 (see also FIGS. 3, 5, and 8).

As also shown in FIG. 1, is one preferred embodiment of a hindquarters unit 30 having a portion for covering the canine or feline rear 31, a securing portion 32 positioned over the hindquarters and a tail bore 44 (see FIG. 6) to allow the animal's tail to pass through said hindquarters unit 30. Further shown are removably securing portions 41 or 42 (see also FIGS. 6 and 8).

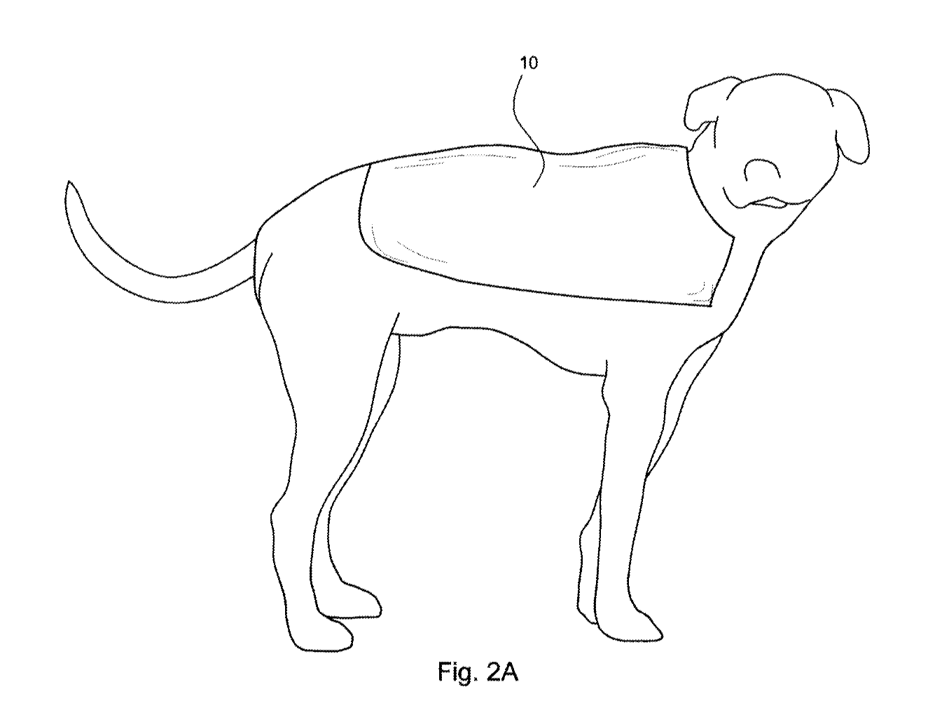

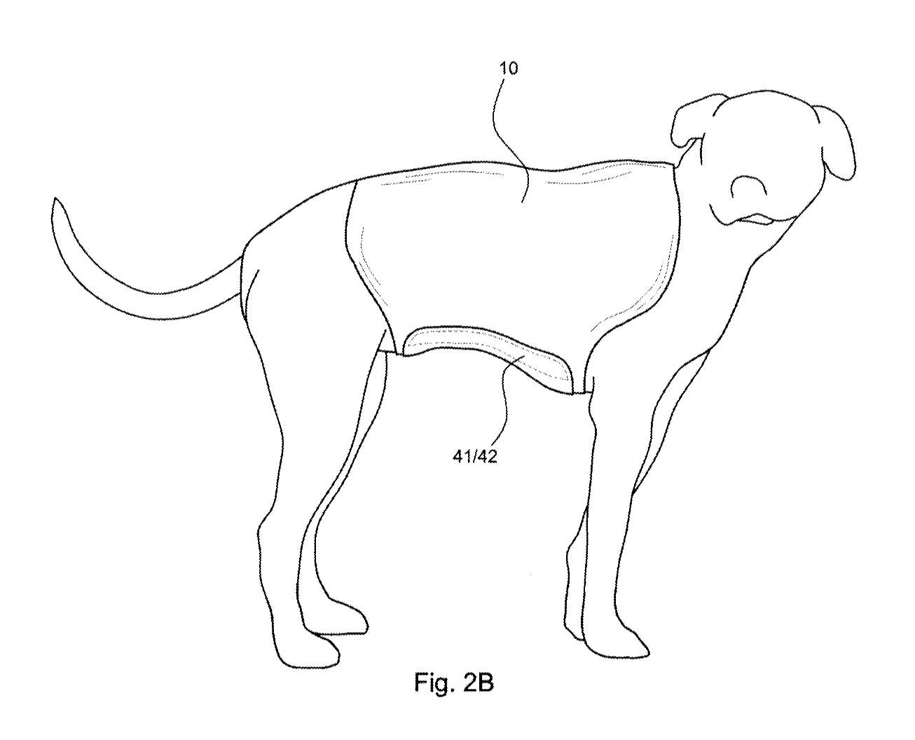

FIG. 2A there is shown a right side view of a preferred embodiment non-self-securing of center unit 10 dimensioned to lay on the back of a canine or feline similarly to a saddle blanket on an equine.

Referring now to FIG. 2B there is shown a right side view of another preferred embodiment of a center unit 10 dimensioned to wrap completely around a canine or feline midsection and having removably securing portion 41 or 42 (see FIG. 8).

Turning now to FIG. 3 there is shown a right side view of a preferred embodiment of a self-securing chest unit 20 mounted on a canine having a chest covering portion 24, a chest front securing portion 21 and a chest rear securing section 22 as well as removably securing portions 41 or 42 (see FIG. 8).

In FIG. 4 there is shown right side view of a preferred embodiment of a self-securing hindquarters unit 30 mounted on a canine having a portion for covering the canine or feline rear 31, a securing portion 32 positioned over the hindquarters and a tail bore 44 (see FIG. 6) to allow the animal's tail to pass through said hindquarters unit 30. Further shown are removably securing portions without finger pockets 41 or with finger pockets 42 (see FIG. 8),

Referring to FIG. 5 there is shown a plan top view of one preferred embodiment of a chest unit 20 comprising a chest covering portion 23 and a chest covering mounting portion 24 allowing for securing to said center unit 10 (see FIGS. 2A and 2B) by means of removably securing portions 41/42 located on the ends 22 and 21 respectively of chest covering portion 23 and chest covering mounting portion 24. Also shown is an optional removably attachable sleeve 25 for receiving hot or cold packs. Said hot or cold packs suitable for use to warm or cool the canine or feline or to provide heat/cold therapy to assist with injury recovery. Said hot and cold packs are well known in the health and fitness arts and need no further description herein. In the context of this invention, hot packs, cold packs, and the like as well as medical sponges, and the like are also referred to as “therapeutic articles.” It is to be understood that the term “sleeve” means a separate pocket, sleeve, or other receiving device dimensioned for holding a desired hot or cold pack. The presently preferred sleeves are manufactured from the same four-way stretch fabric materials having an unbroken loop (UBL) fabric on the both exterior surfaces with an open cell polyurethane center layer sandwiched between. One exterior surface of said sleeve having a low profile hook portion of a hook and loop tape disposed thereon, thus allowing the removable attachment of said sleeve in any desired location.

Turning now to FIG. 6 there is shown a non-self-securing hindquarters unit 30 suitable for use as part of said veterinary modular body wrap 100, comprising a portion for covering the canine or feline rear 31, a securing portion 32 positioned over the hindquarters and a tail bore 44 to allow the animal's tail to pass through said hindquarters unit 30. Further shown are removably securing portions without finger pockets 41 or with finger pockets 42.

Turning now to FIG. 7 there is shown a plan top view of a preferred embodiment of a non-self-securing center unit 10 suitable for use as part of said claimed veterinary modular body wrap 100.

In FIG. 8 there is shown a perspective end view of a securing portion 42 of modular units 10, 20, and 30 comprising the fabric of said modular units 10, 20, and 30 and a piece of additional material permanently fixed to said modular unit fabric along three of four edges. The fourth edge being along the corresponding edge of said modular unit fabric thereby creating a pocket suitable for inserting a finger to assist in pulling said securing portion 42 and detaching units of said claimed veterinary modular body wrap 100. It to be understood that well known means of securing in the art such as sewing with thread, heat bonding, and adhesives, for example, are suitable for mounting said additional material. It is to be further appreciated that said additional material is a substantially non-stretch material well known in the art.

The term “finger pockets” as used herein mean an opening at the end of a securing portion 42 which has an opening created between the modular unit fabric and the hook portion of a hook-and-loop connector allowing a person to insert a finger between the two layers to more easily allow un-securing the connection without the need for pull tabs.

It is to be further understood that a “non-self-securing” modular unit requires the use of a center modular unit for securely mounting the “non-self-securing” modular unit on a feline or canine and that a self-securing modular unit doesn't require the use of a center modular unit.

One particularly preferred embodiment of the present invention provides for a veterinary modular body wrap comprising three modular units for positioning and holding a medical dressing or therapeutic article in at least one desired location on an animal body comprising in cooperative combination: a modular center unit dimensioned to lay across the back of an animal body comprising four-way stretch fabric materials having an unbroken loop (UBL) fabric on the both exterior surfaces with an open cell polyurethane center layer sandwiched between; a modular chest unit dimensioned to provide a chest covering portion comprising four-way stretch fabric materials having an unbroken loop (UBL) fabric on the both exterior surfaces with an open cell polyurethane center layer sandwiched between, removably attachable to said center unit by modular chest unit attachment portions having removably securing portions comprising the hook part of a hook-and-loop connection; and a modular hindquarters unit dimensioned for covering the canine or feline hindquarters comprising four-way stretch fabric materials having an unbroken loop (UBL) fabric on both exterior surfaces with an open cell polyurethane center layer sandwiched between, removably attachable to said center unit by securing portion positioned over the hindquarters comprising the hook part of a hook-and-loop connection, and a tail bore allowing the animal's tail to pass through said modular hindquarters unit; thereby creating a desired modular body wrap consisting comprising three modular units for positioning and holding a medical dressing or therapeutic article in desired positions on an animal's body.

Another particularly preferred embodiment of the present invention provides for a veterinary modular body wrap comprising two modular units for positioning and holding a medical dressing or therapeutic article in at least one desired location on an animal body comprising in cooperative combination: a modular chest unit dimensioned to provide a chest covering portion comprising four-way stretch fabric materials having an unbroken loop (UBL) fabric on both exterior surfaces with an open cell polyurethane center layer sandwiched between, removably attachable to said animal body by modular chest unit attachment portions having removably securing portions comprising the hook part of a hook-and-loop connection; and a modular hindquarters unit dimensioned for covering the canine or feline hindquarters comprising four-way stretch fabric materials having an unbroken loop (UBL) fabric on both exterior surfaces with an open cell polyurethane center layer, removably attachable said animal body by securing portion positioned over the hindquarters comprising the hook part of a hook-and-loop connection, and a tail bore allowing the animal's tail to pass through said modular hindquarters unit; thereby creating a desired modular body wrap consisting comprising two modular units for positioning and holding a medical dressing or therapeutic article in desired positions on an animal's body.

The presently preferred veterinary modular body wrap material promotes the transfer of air and moisture to and from the wound site to help promote healing. Particularly preferred, is a four-way stretch fabric materials having an unbroken loop (UBL) fabric on both exterior surfaces with an open cell polyurethane center layer. Said (UBL) exterior surface fabric acting as the loop portion of a hook and loop attachment system.

The presently preferred securing means 7 is a low profile hook and loop tape particularly Velcro brand from Velcro USA of Manchester, N.H. Especially preferred is the use of Velcro hook material and unbroken loop (UBL) exterior fabric surface of said veterinary dressing wrap material forming the securing means of the present invention.

The presently preferred gauze is gauze made of 30% polyester and 70% viscose manufactured by Kingphar Medical Material Co., Ltd of Hubei, China. The presently preferred adhesive films are double coated pressure sensitive adhesive films comprise a thin polyester film double coated with removable acrylic adhesive on one side and a high performance permanent rubber adhesive on the other side, manufactured by Scapa North America in Windsor, CT. The most preferred veterinary dressing or medical sponge comprises a multiple layer material having sterile gauze covered on a first surface/side by a polyethylene (PE) film wherein the film is perforated to allow passage of air and moisture into the sterile gauze material and a polyester film double coated with a removable acrylic adhesive on the second surface/side of said medical dressing. The adhesive on the first surface/side attaching the polyester film to the sterile gauze material and the second surface/side covered by a removable adhesive protecting layer which is removed before the medical dressing is removably securable to said dressing wrap material. Preferably the adhesives used are latex free, and can be of any suitable formulation. The presently preferred gauze comprises 30% polyester and 70% viscose fabric.

The various components of the present invention are preferably sewn together using nylon or Dacron thread using well known techniques in the sewing arts. However, it is to be appreciated that the manner of attachment of the various components to one another may be by any suitable well known method, such as, for example, adhesives, heat welding, sonic welding, and the like. It is further to be appreciated that while the present invention provides flexibility in fitting to various sized animals this can be further enhanced as to fit and comfort by providing said modular body wrap of the present invention in a variety of physical sizes.

Although the preferred embodiments of the present invention has been disclosed, various changes and modifications may be made without departing from the scope of the invention as set forth in the appended claims.

What is claimed is:

1. A veterinary modular body wrap comprising three modular units for positioning and holding at least one medical dressing or therapeutic article in at least one desired location on an animal body comprising in cooperative combination:
a modular center unit dimensioned to lay across the back of an animal body comprising four-way stretch fabric materials having an unbroken loop (UBL) fabric on the both exterior surfaces with an open cell polyurethane center layer sandwiched between;
a modular chest unit dimensioned to provide a chest covering portion comprising four-way stretch fabric materials having an unbroken loop (UBL) fabric on the both exterior surfaces with an open cell polyurethane center layer sandwiched between; and
a modular hindquarters unit dimensioned for covering the canine or feline hindquarters comprising four-way stretch fabric materials having an unbroken loop (UBL) fabric on both exterior surfaces with an open cell polyurethane center layer sandwiched between, removably attachable to said center unit by securing portion positioned over the hindquarters comprising the hook part of a hook-and-loop connection, and a tail bore allowing the animal's tail to pass through said modular hindquarters unit; thereby creating a desired modular body wrap consisting comprising three modular units for positioning and holding a medical dressing or therapeutic article in desired positions on an animal's body.

2. The veterinary modular body wrap as claimed in claim 1 wherein, said therapeutic article comprises at least one cold pack removably attached using hook and loop attachment in at least one desired location.

3. The veterinary modular body wrap as claimed in claim 1 wherein, said therapeutic article comprises at least one hot pack removably attached using hook and loop attachment in at least one desired location.

4. The veterinary modular body wrap as claimed in claim 1 wherein, said at least one medical dressing is removably attached using hook and loop attachment in at least one desired location.

5. A veterinary modular body wrap comprising two modular units for positioning and holding a medical dressing or therapeutic article in at least one desired location on an animal body comprising in cooperative combination:
a modular chest unit dimensioned to provide a chest covering portion comprising four-way stretch fabric materials having an unbroken loop (UBL) fabric on both exterior surfaces with an open cell polyurethane center layer sandwiched between, removably attachable to said animal body by modular chest unit attachment portions having removably securing portions comprising the hook part of a hook-and-loop connection; and
a modular hindquarters unit dimensioned for covering the canine or feline hindquarters comprising four-way stretch fabric materials having an unbroken loop (UBL) fabric on both exterior surfaces with an open cell polyurethane center layer, removably attachable said animal body by securing portion positioned over the hindquarters comprising the hook part of a hook-and-loop connection, and a tail bore allowing the animal's tail to pass through said modular hindquarters unit;
thereby creating a desired modular body wrap consisting comprising two modular units for positioning and holding a medical dressing or therapeutic article in desired positions on an animal's body.

6. The veterinary modular body wrap as claimed in claim 5 wherein, said therapeutic article comprises at least one cold pack removably attached using hook and loop attachment in at least one desired location.

7. The veterinary modular body wrap as claimed in claim 5 wherein, said therapeutic article comprises at least one hot pack removably attached using hook and loop attachment in at least one desired location.

8. The veterinary modular body wrap as claimed in claim 5 wherein, said at least one medical dressing is removably attached using hook and loop attachment in at least one desired location.
